(19) United States
(12) Patent Application Publication
(10) Pub. No.: US 2026/0324992 A1
(43) Pub. Date: Oct. 22, 2026

(54) ADJUSTABLE BLOOD PRESSURE CUFF

(71) Applicant: ORLANDO HEALTH, INC., Orlando, FL (US)

(72) Inventors: Elisabeth Ashley Gleason, North Port, FL (US); Tyler Fernandes Goncalves, Saint Johns, FL (US); Caleb John McCue, Sarasota, FL (US); Daniel Thomas O'Brien, Ponce Inlet, FL (US); Jared Brenden Olitsky, Ft. Myers, FL (US); Evan Cuellar Gomez, Orlando, FL (US)

(21) Appl. No.: 19/649,702

(22) Filed: Apr. 16, 2026

Related U.S. Application Data
(60) Provisional application No. 63/790,466, filed on Apr. 17, 2025.

(51) Int. Cl. A61B 5/022; A61B 5/021; A61B 5/0235

(52) U.S. Cl. CPC A61B 5/02233; A61B 5/02141; A61B 5/0235

(57) ABSTRACT

A blood pressure measuring device comprises a cuff including multiple segments arranged along a first direction. Within these segments, there are a plurality of air bladders that can be selectively inflated, allowing the length of the inflated portion of the air bladders in the first direction to be adjusted. Additionally, the blood pressure measuring device includes a clamp that can be selectively attached to a portion of the cuff to prevent air flow. The configuration of the clamp and the multiple segments allow the circumference of the inflated portion of the air bladders to be adjusted when the cuff is wrapped around a patient's arm.

### PRIORITY CLAIM AND CROSS-REFERENCE TO RELATED APPLICATIONS

This application claims priority claims priority U.S. Provisional Patent Application No. 63/790,466, filed Apr. 17, 2025, and entitled “ADJUSTABLE BLOOD PRESSURE CUFF,” which is hereby incorporated herein by reference in its entirety to be considered part of this specification.

### BACKGROUND

The present disclosure relates generally to non-invasive blood pressure monitoring, and in particular, a blood pressure cuff that can be adjusted to the patient to provide blood pressure measurement with increased accuracy.

In 2022, high blood pressure was a primary contributing cause of 685,875 deaths in the United States alone. Furthermore, blood pressure medications are designed to reduce high blood pressure, but in people without hypertension, these drugs could lower blood pressure too much, potentially leading to hypotension. Symptoms of hypotension include dizziness, fainting, fatigue, blurred vision, and even shock in severe cases. Nearly half of adults accounted for in America are diagnosed with hypertension, and out of those, only about a quarter have their hypertension under control. Moreover, millions of Americans are misdiagnosed every year.

Blood pressure measurements rely on detecting systolic and diastolic pressure via occlusion of the brachial artery using an inflatable air bladder inside a blood pressure cuff. The brachial artery is preferred due to its accessibility and reliability. Alternatives, such as the radial artery and femoral artery, present limitations in accuracy and usability.

Blood pressure monitoring systems can be either manual or automatic. Manual blood pressure monitoring systems consist of a bulb pump and an air valve, which are used to inflate the air bladder and a stethoscope is used by the operator to detect Korotkoff sounds. Blood pressure monitors can also be automatic, which are also referred to as electric systems. These electric systems consist of an electric pump that inflates the air bladder and include an oscillometric sensor to detect pressure.

For accurate readings, a blood pressure cuff's air bladder must cover at least 80% of the arm circumference and at least 40% of the upper arm length. This sizing is crucial to avoid incorrect blood pressure measurements due to improper air bladder sizing.

However, conventional blood pressure cuffs designed to aid in these diagnoses are not one-size-fits-all devices. Blood pressure cuffs may provide incorrect measurements if they are not properly fitted to the patient. Poorly fitted blood pressure cuffs can cause systolic measurement errors as low as 4 millimeters of mercury (mmHg), and as high as 19.5 mmHg. These differences in blood pressure could falsely suggest that a patient has normal blood pressure when they really need to seek medical attention. Thus, inaccurate blood pressure measurements can put patients at serious risks.

To obtain an accurate blood pressure measurement, the operator must find a blood pressure cuff with the correct air bladder size. However, current blood pressure cuffs have an air bladder with a fixed geometry.

Further, blood pressure cuffs should include the following properties: adjustable sizing to accommodate a wide range of arm sizes without compromising accuracy; hypoallergenic and biocompatible materials; materials much be able to withstand disinfectants; the blood pressure cuff must inflate and deflate consistently; the device should require minimal operator training; and the blood pressure cuff must be compatible with standard blood pressure monitoring systems.

Thus, there exists a need for an adjustable blood pressure cuff, specifically, a blood pressure cuff with adjustable air bladder sizing, to ensure that the bladder is able to be adjusted to the correct size for a variety of patients. A need further exists for an adjustable blood pressure cuff that also includes biocompatible materials, environmental resistant materials, inflation system reliability, user friendliness, and compatibility as noted above. An adjustable blood pressure cuff with the above listed features will aid in preventing misdiagnosis stemming from blood pressure measurements. With an easily usable and adjustable blood pressure cuff, patients will be able to receive care that is truly tailored to their condition.

### SUMMARY

The present disclosure involves the use of a blood pressure cuff assembly with multiple air bladders and a corresponding controllable tubing system to allow for adjustment based on arm size.

The blood pressure measuring device in one embodiment includes a cuff including a plurality of segments disposed along a first direction; a plurality of air bladders disposed in the plurality of segments, wherein the plurality of air bladders are configured to be selectively inflated such that a length of an inflated portion of the plurality of air bladders in the first direction is adjustable; and a flow control device configured to be selectively coupled to a portion of the cuff, wherein the flow control device is configured to prevent air flow such that a circumference of the inflated portion of the plurality of air bladders is adjustable when the cuff is wrapped around an arm of a patient.

In a second aspect of the present disclosure, which may be combined with any other aspect, or portion thereof, the blood pressure measuring device includes a tubing system configured to selectively provide air to the plurality of air bladders.

In a third aspect of the present disclosure, which may be combined with any other aspect, or portion thereof, the tubing system includes a plurality of valves configured to selectively allow or prevent airflow to the plurality of air bladders, respectively.

In a fourth aspect of the present disclosure, which may be combined with any other aspect, or portion thereof, the plurality of valves includes a stopcock.

In a fifth aspect of the present disclosure, which may be combined with any other aspect, or portion thereof, the plurality of air bladders includes a first air bladder and a second air bladder, and wherein the first air bladder has a greater length in the first direction than the second air bladder.

In a sixth aspect of the present disclosure, which may be combined with any other aspect, or portion thereof, each segment of the plurality of segments of the cuff is configured to limit a height and a length in the first direction of a respective air bladder of the plurality of air bladders disposed therein when inflated.

In a seventh aspect of the present disclosure, which may be combined with any other aspect, or portion thereof, the plurality of valves includes a T connector.

In an eighth aspect of the present disclosure, which may be combined with any other aspect, or portion thereof, a length of the tubing system is at least 8.31 inches.

In a ninth aspect of the present disclosure, which may be combined with any other aspect, or portion thereof, the flow control device is configured to compress the cuff to block airflow within the plurality of air bladders beyond the portion of the cuff to which the flow control device is coupled.

In a tenth aspect of the present disclosure, which may be combined with any other aspect, or portion thereof, the flow control device is movable along the first direction to select the circumference of the inflated portion.

In an eleventh aspect of the present disclosure, which may be combined with any other aspect, or portion thereof, the flow control device is a clamp and the clamp includes opposing clamp members configured to engage opposite sides of the cuff.

In a twelfth aspect of the present disclosure, which may be combined with any other aspect, or portion thereof, the cuff includes an outer fabric layer configured to house the plurality of air bladders.

In a thirteenth aspect of the present disclosure, which may be combined with any other aspect, or portion thereof, a polymer material comprises a thermoplastic polyurethane-coated fabric.

In a fourteenth aspect of the present disclosure, which may be combined with any other aspect, or portion thereof, the flow control device and selective inflation together define a two-dimensional adjustment of the inflated portion of the cuff.

In a fifteenth aspect of the present disclosure, each of the plurality of segments has a length in the first direction of about 1 cm to about 3 cm.

In a sixteenth aspect of the present disclosure, which may be combined with any other aspect, or portion thereof, each of the plurality of segments has a length of about 2 cm.

In a seventeenth aspect of the present disclosure, which may be combined with any other aspect, or portion thereof, the inflated portion of the plurality of air bladders has an adjustable circumference of about 20 cm to about 35 cm when wrapped around the arm of the patient.

In an eighteenth aspect of the present disclosure, which may be combined with any other aspect, or portion thereof, the inflated portion of the plurality of air bladders is configured to cover at least about 80% of a circumference of the patient's arm.

In a nineteenth aspect of the present disclosure, which may be combined with any other aspect, or portion thereof, the clamp has a length in the first direction of about 25 cm to about 40 cm.

In a twentieth aspect of the present disclosure, which may be combined with any other aspect, or portion thereof, the clamp has a length of about 36 cm.

In light of the above aspects and the present disclosures set forth herein, it is accordingly an advantage of the present disclosure to provide a blood pressure cuff design that has positive economic impacts. Specifically, the subject design is in the form of reducing medical device waste. For example, instead of clinicians retaining multiple blood pressure cuffs of different sizes, healthcare offices can simply hold one type, the new adjustable blood pressure cuff design, thus reducing the overproduction and underutilization of certain existing blood pressure cuff sizes.

Additional features and advantages are described in, and will be apparent from, the following Detailed Description and the Figures. The features and advantages described herein are not all-inclusive and, in particular, many additional features and advantages will be apparent to one of ordinary skill in the art, in view of the figures and description. Also, any particular embodiment does not have to have all of the advantages listed herein, and it is expressly contemplated to claim individual advantageous embodiments separately. Moreover, it should be noted that the language used in the specification has been selected principally for readability and instructional purposes, and not to limit the scope of the inventive subject matter.

### BRIEF DESCRIPTION OF THE FIGURES

FIG. 1 is a side view of one embodiment of the air bladder of the present disclosure.

FIG. 2 is a side view of one embodiment of the air bladder of the present disclosure.

FIG. 3 is a perspective view of one embodiment of a connection port of the present disclosure.

FIG. 4 is a diagram of an embodiment of the tubing system of the present disclosure.

FIG. 5A is a perspective view of one embodiment of a first half of a top clamp piece of the present disclosure.

FIG. 5B is a perspective view of one embodiment of a first half of a bottom clamp piece of the present disclosure.

FIG. 5C is a perspective view of one embodiment of a second half of a bottom clamp piece of the present disclosure.

FIG. 6A is a perspective view of one embodiment of a first half of a clamp pin of the present disclosure.

FIG. 6B is a perspective view of one embodiment of a second half of a clamp pin of the present disclosure.

FIG. 7 is a perspective view illustrating one embodiment of a clamp of the present disclosure.

FIG. 8A is a side view illustrating one embodiment of an outer cuff front panel on its non-TPU coated side of the present disclosure.

FIG. 8B is a side view illustrating one embodiment of an outer cuff back panel on its non-TPU coated side of the present disclosure.

FIG. 9A is a side view illustrating one embodiment of an outer cuff front panel on its TPU coated side of the present disclosure.

FIG. 9B is a side view illustrating one embodiment of an outer cuff back panel on its TPU coated side of the present disclosure.

### DETAILED DESCRIPTION

In certain embodiments, a blood pressure cuff assembly is provided comprising a plurality of structural components configured to cooperate for controlled occlusion and measurement. The assembly may include an inflatable air bladder disposed within an outer enclosure formed of nylon or other durable textile material. The enclosure is configured to retain the air bladder while providing flexibility and resistance to wear during repeated use. The assembly further comprises an adjustable securing mechanism, such as one or more hook-and-loop fasteners, configured to releasably engage and maintain the cuff in a desired position about a patient's limb. In addition, the assembly includes a fluid conduit, which in some embodiments comprises polyvinyl chloride (PVC) or non-PVC tubing, operatively coupled to the air bladder and configured to place the air bladder in fluid communication with a pressure control system.

In some embodiments, the inflatable air bladder may be configured for use with a variety of inflation mechanisms. For example, manual inflation systems may include a compressible bulb pump configured to introduce air into the bladder, such systems being associated with high measurement accuracy in clinical settings. In other embodiments, the cuff assembly may be operatively coupled to an electrically powered pump configured to provide automated inflation, such configurations being suitable for home-use or self-monitoring applications. In further embodiments, the inflation source may comprise a compressed gas cartridge, such as a carbon dioxide (CO₂) cartridge, configured for single-use or emergency deployment scenarios, including but not limited to personal flotation devices or inflatable safety equipment.

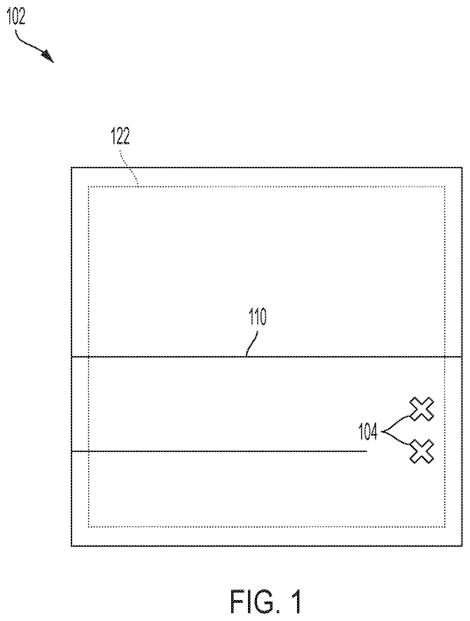

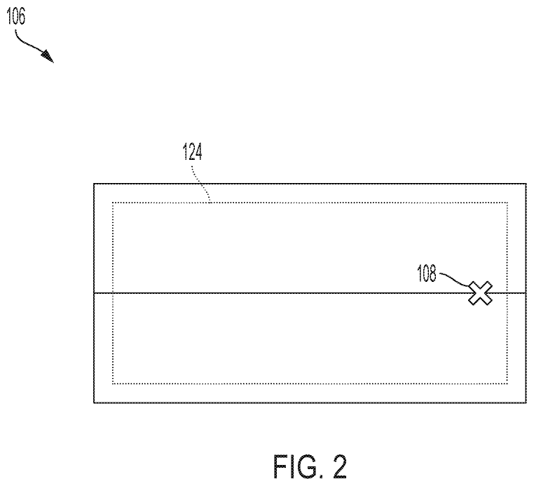

Referring now to the drawings and, in particular, to FIGS. 1 to 8. FIG. 1 and FIG. 2 provide a side view of one embodiment of the air bladder of the present disclosure. The air bladder provided in FIG. 1 is a large air bladder 102 and the air bladder provided in FIG. 2 is a small air bladder 106.

The blood pressure cuff of the present disclosure contains five air bladders: one large air bladder 102 and four small air bladders, such as the air bladder 106 provided in FIG. 2. The large air bladder 102 and the small air bladder 106 may be constructed from 70D TPU coated woven nylon to ensure biocompatibility. This material is advantageously lighter weight and lower cost in comparison to thicker materials. Thus, the large air bladder 102 and the small air bladder 106 are configured to be included in the blood pressure cuff according to the present invention.

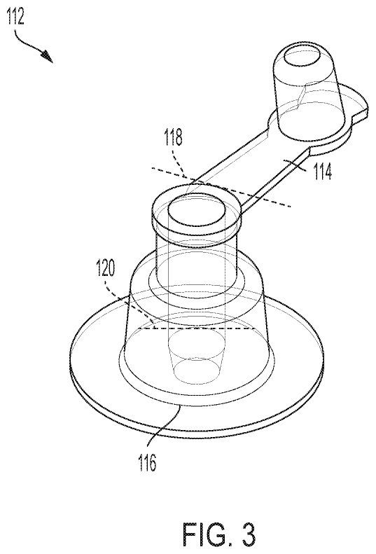

The large air bladder 102 includes at least one hole 104 and the small air bladder 106 includes a hole 108. The at least one hole 104 and the hole 108 are configured to allow for engagement between a connection port 112 (see FIG. 3). In an example embodiment, the at least one hole 104 and the hole 108 are created from cuts in the material of their respective air bladder. In an example embodiment, at least six total connection ports may be engaged to the blood pressure cuff via the large or small air bladders 102, 106. In another example embodiments, the holes 104, 108 are made from X shaped cuts in their respective air bladders.

In an example embodiment, the large air bladder 102 has a first end, a second end, and a partial seal 110 located at a distance from the first end and at a distance from the second end. In a preferred embodiment, the distance from the first end and the distance from the second end are equal. In other words, the seal 110 bisects a middle section of the large air bladder 102. The seal 110 does not cut off airflow. Rather, the seal 110 controls an inflated height to reach 4 cm. This creates an even pressure application on the arm.

The partial seal 110 of the large air bladder 102 is located, in an example embodiment, 12 cm from the first end and 12 cm from the second end. Various distances may be appreciated. Thus, the total width of the large air bladder, in the example embodiment of FIG. 1, is 24 cm and the length is 39 cm. The large air bladder 102 may also contain a second seal located at a distance between the seal 110 and either the first end or the second end. The second seal, in one example embodiment, is 30 cm and rests parallel to the seal 110. In an example embodiment, the second seal is located parallel to the seal 110 at a distance of 6 cm from the seal 110.

The at least one hole 104 of the large air bladder 102 may be two holes, wherein one hole is located 6 cm from a first end or a second end and a second hold is located 4 cm from a first hole. The at least one hole 104 is located 4 cm from an edge of the large air bladder 102. The large air bladder 102 further includes a seam 112, which is located 2 cm from an edge of the large air bladder 102.

Turning to FIG. 2, the small air bladder 106 includes a width of 14 cm and a length of 39 cm. The small air bladder 106 includes a seal that bisects the width of the small air bladder 106. In the example embodiment disclosed in FIG. 2, the seal is located 7 cm from a first end and 7 cm from a second end. The hole 108 of the small air bladder rests on the seal at a location of about 4 cm away from an edge of the small air bladder 106. The small air bladder 106 includes a seam located 2 cm from an edge of the small air bladder 106.

### Air Bladder Manufacture

The first step is preparing the materials. Nylon sections should be cut for both the large air bladders 102 and the small air bladders 106. Additionally, small holes will be cut in the large air bladder 102 and the small air bladder 106 for the at least one hole 104 and the hole 108. These holes 104, 108 are configured to engage with a connection port 112, such as the connection port 112 illustrated in FIG. 3. The connection port 112 includes a top cap 114 portion and a one way valve 116. The cap 114 portion is figured to be cut on a line 118, provided in FIG. 3 for illustrative purposes only. Further, the connection port 112 is configured to be cut at line 120, provided in FIG. 3 for illustrative purposes only, which is a section that acts as a one way valve.

A connection port 112 is then inserted into the holes 104, 108 of the air bladder, such as air bladders 102, 106. The connection ports 112 will be inserted starting from a side of the nylon coated in thermoplastic polyurethane (TPU), which will be described in further detail below. This allows the small rim around the valve to be radio-frequency (RF) welded to the nylon. RF welding is selected as the preferred manufacturing method, as it creates a strong bond, without the need for additional adhesives, and is efficient.

The next step is to RF weld the seams of the five air bladders. As illustrated in FIG. 1, the large air bladder 102 includes a seam 122 with a 2 cm seal thickness. As illustrated in FIG. 2, the small air bladder 106 includes a seam 144 with a 2 cm seal thickness. To seal the air bladders, the nylon material will be folded in half and sealed along the remaining three edges. One additional step is taken for the large air bladder 102. The second seal shown in FIG. 1 is an additional seal, whose purpose is to limit the inflation height of the large air bladder. This is added by heat sealing along the second seal for a thickness of approximately 0.5 cm.

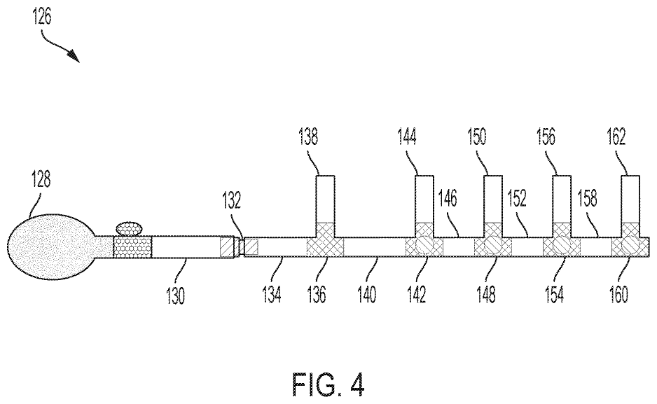

Last, a barbed connection fitting is placed into each of the six connection ports 112, so a tubing system, such as the tubing system 126 of FIG. 4, can be later connected. Before the air bladders 102, 106 are incorporated into the outer cuff, quality control is vital to ensure there are no leaks in the air bladders.

FIG. 4 includes a diagram of an embodiment of the tubing system 126 of the present disclosure. The tubing system 126 includes tubing 130, 134, 140, 146, 152, 158, 138, 144, 150, 156, and 162. The tubing system 126 further includes a bulb pump 128, a barb reducer fitting 132, a T connector 136, and stopcocks 142, 148, 154, 160.

The total internal length of the tubing system 126 from the T connector 136 to the stopcock 160 is 7.133 in, compared with the 7.087 in in available length along the air bladders. While slightly longer, the length of the tubing system 126 is close to this available length. This allows the valves to be closely aligned to their corresponding air bladders, reducing the length of connective tubing needed. The total length of the tubing system 126 is 36.78 in, from the bulb pump 1 to the stopcock 160, with the majority of the length coming from the tubing 130 that connects the bulb pump 128 held by the operator to the blood pressure cuff itself.

A benefit of the illustrated tubing system 126 over other conventional systems is the switch from the multiport control lever valve to a series of stopcocks 142, 148, 154, 160. The stopcocks 142, 148, 154, 160 are biocompatible and are commonly used in IV lines, allowing for increased user friendliness of the design, as the operator, a healthcare professional, typically has experience with stopcocks. Within the design, the stopcocks 142, 148, 154, 160 work to control the flow of air both to the air bladders and the tubing system 126.

As shown in FIG. 4, the stopcocks 142, 148, 154, 160 are of the slip luer design, meaning the tubing, such as the illustrating tubing 130, 134, 140, 146, 152, 158, 138, 144, 150, 156, 162, slips over the connection points of the stopcocks 142, 148, 154, 160 so no additional components are needed. Thus, the length of tubing 130, 134, 140, 146, 152, 158, 138, 144, 150, 156, 162 between each stopcock can be minimized by placing the ends of two stopcocks next to each other without any additional tubing space in between. This is how the length of tubing needed for component number 4, 7, 10, 13, and 16 was determined.

Further, the length of tubing required to connect the tubing system 126 to the air bladders 102, 106 is the same length to reduce the manufacturing cost of having five different tubing lengths. Table 1 provides the horizontal and vertical distances between the end of the T connector or one of the stopcocks used in the tubing system and the connection port on the air bladders. These values were used to find the tubing length needed between these sections, in addition to the 0.5 in length on either side used to connect the tubing both to the valve and the connection port. These lengths were determined by lining up the tubing system layout with the air bladder layout and measuring the distances between the centers of the valves and the centers of the T connector or one of the stopcocks. From Table 1, it is seen that the largest distance needed is 2.52 in, and including the 1 in additional distance needed, the total tubing distance is 3.52 in.

TABLE 1
Tubing Length

| | Horizontal | Vertical | Tubing |
|---|---|---|---|
| | Distance | distance | Length |
| Reference | (in) | (in) | (in) |
| T connector 136 | 1 | 1.33 | 1.66 |
| Stopcock 142 | 1 | 2.31 | 2.52 |
| Stopcock 148 | 1 | 1.52 | 1.82 |
| Stopcock 154 | 1 | 0.83 | 1.30 |
| Stopcock 160 | 1 | 0.14 | 1.01 |

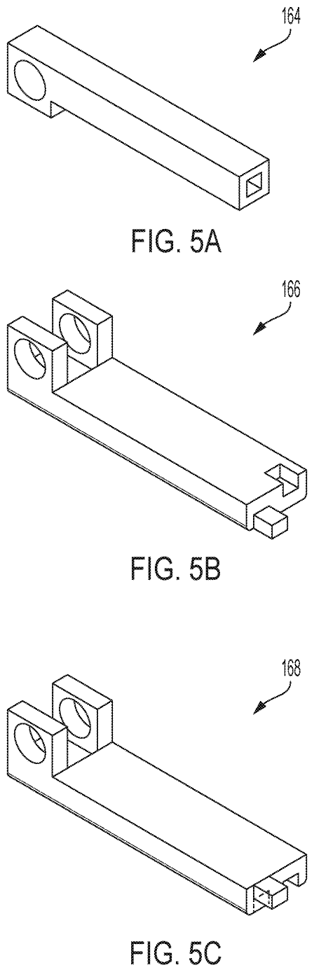

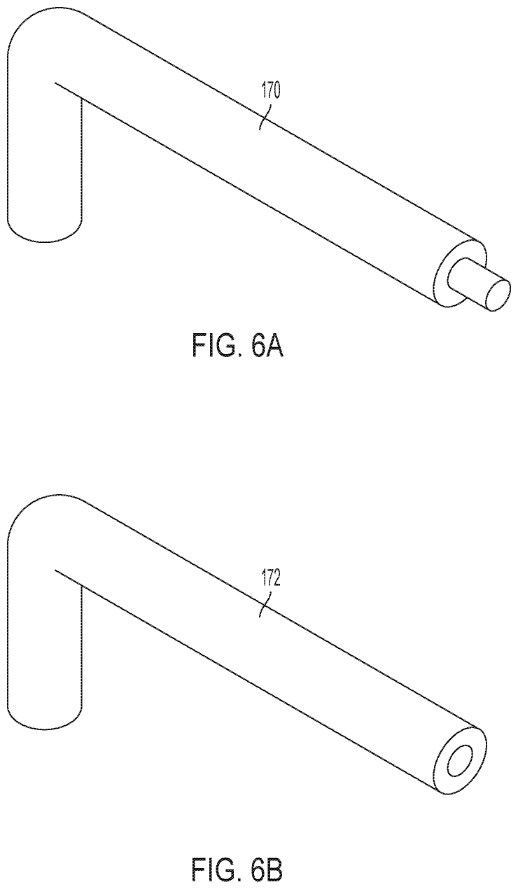

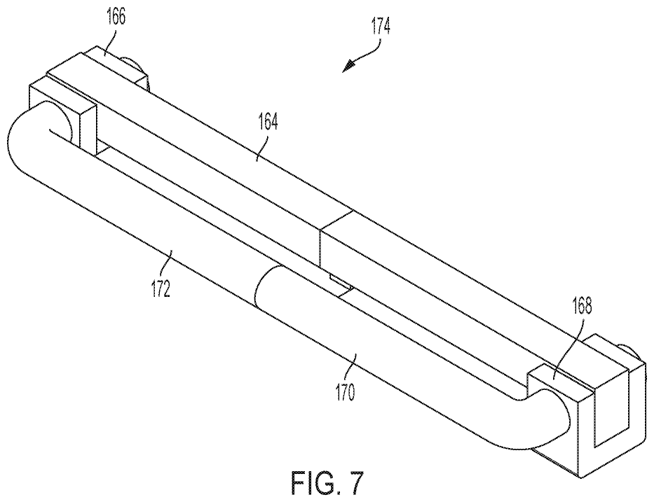

FIG. 5A is a perspective view of one embodiment of a first half of a top clamp piece 164 of the present disclosure. FIG. 5B is a perspective view of one embodiment of a first half of bottom clamp piece 166 of the present disclosure. FIG. 5C is a perspective view of one embodiment of a second half of bottom clamp piece 168 of the present disclosure. FIG. 6A is a perspective view of one embodiment of a first half of a pin 170 of the present disclosure. FIG. 6B is a perspective view of one embodiment of a second half of a pin 172 of the present disclosure. FIG. 7 is a perspective view illustrating one embodiment of a clamp 174 comprising the first half of a top clamp piece 164 of FIG. 5A, the first half of bottom clamp piece 166 of FIG. 5B, the second half of bottom clamp piece 168 of FIG. 5C, the first half of a pin 170 of FIG. 6A, and the second half of a pin 172 of FIG. 6B of the present disclosure.

The clamp 174 consists of three main components: a top clamp piece, a bottom clamp piece, and a clamp pin. To create the final clamp, each of the components illustrated below would be injection molded as solid pieces.

In some example embodiments, the first half of a top clamp piece 164 includes a first end and a second end. The first end of the top clamp piece 164 includes a width that is 3 cm. The second end of the top clamp piece 164 includes a width that is 2 cm. The length of the top clamp piece 164 may be about 16 cm. Other dimensions are appreciated herein. The first end of the top clamp piece includes a hole configured to engage with a pin. The hole may have a diameter less than 3 cm. Moreover, the top clamp piece 164 includes a hollow interior. The hollow interior runs along the length of the top clamp piece 164 and includes a length of 1 cm and a width of 1 cm.

Referring now FIG. 5B, in an example embodiment, a first half of a bottom clamp piece 166 is illustrated. The first half of a bottom clamp piece 166 as provided includes an elongated body. The elongated body may generally be of a rectangular configuration and is configured to mate with first half of a top clamp piece 164 and a second half of bottom clamp piece 168, such as those provides in FIGS. 5A and 5C, respectively.

In the illustrated embodiment of FIG. 5B, the first half of a bottom clamp piece 166 includes an elongated body having a length of approximately 17.25 cm, with a main straight section extending approximately 13.00 cm from a rear mounting region toward a forward end. In one example, the main straight section of the longitudinal body portion has a thickness of approximately 1.25 cm and a width of approximately 4.00 cm, although other dimensions may be used.

At a rear end of the first half of a bottom clamp piece 166, a mounting portion is provided, including an opening configured to receive a fastener or pivot element. In some example embodiments, the opening may be circular. In the illustrated example, the circular opening has a diameter of approximately 2.05 cm. The mounting portion extends upward from the main body and includes vertical wall surfaces having heights of approximately 4.03 cm and 3.73 cm, as illustrated. A transition region between the mounting portion and the longitudinal body includes a filleted or radiused surface having a radius of approximately 0.30 cm.

The longitudinal body of the first half of bottom clamp piece 166 further includes an upper recess or channel extending along at least a portion of its length. In the illustrated embodiment, the channel has a width of approximately 2.00 cm and a depth of approximately 2.45 cm, defining opposing side walls. The channel is configured to receive a pin, such as the pins provided in FIGS. 6A and 6B.

At the forward end of the first half of a bottom clamp piece 166, a stepped engagement feature is formed. In the illustrated embodiment, the stepped engagement feature includes a lower projection extending approximately 0.96 cm vertically and approximately 1.00 cm longitudinally, with an adjacent upper projection extending approximately 0.73 cm. These stepped engagement features are configured to interlock with a corresponding feature of the second half of bottom clamp piece 168 of FIG. 5C.

Moving to FIG. 5C, the second half of the bottom clamp piece 168 may include, in an example embodiment, substantially similar dimensions as the first half of the bottom clamp piece 166. However, a longitudinal body of the second half of the bottom clamp piece 168 includes a lower engagement structure configured to interlock with the stepped engagement feature of the first half of a bottom clamp piece 166. In the illustrated example, the forward end of the second half includes a stepped projection having a vertical height of approximately 0.88 units and a longitudinal extension of approximately 1.23 units. A further reduced step portion extends approximately 0.73 units vertically, defining a multi-level engagement profile.

FIGS. 6A and 6B provide example embodiments of a first half of a clamp pin 170 and a second half of a clamp pin 172, respectively. The first half of a clamp pin 170 and the second half of a clamp pin 172 are configured in an L-shape and include a long end and a short end. The long end may include a length of 15.50 cm and the short end may include a length of 5.43 cm. Various dimensions are appreciated herein. The first half of a clamp pin 170 and the second half of a clamp pin 172 include a distal end and a proximal end. The distal end is located at the end the long end of each pin and the proximal end is located at an end of the short end of each pin. The distal end of the first half of a clamp pin 170 includes a protrusion with a diameter that is less than the diameter of the long end, wherein the protrusion is configured to engage with a hollow cavity of the second half of a clamp pin 172. The hollow cavity is located on a distal end of the second half of a clamp pin 172.

It will be understood that the dimensions provided above are illustrative example dimensions and are not intended to be limiting. Variations in size, proportion, and geometry may be made while maintaining the functional relationships of the clamp 174.

Other types of flow restriction devices such as valves or in-line flow restrictors may be used in place of or alongside a clamp to module the air flow in the series of bladders.

TABLE 2
Clamp Components and Volumes

| Section | Name | Volume (cm³) |
|---|---|---|
| Top clamp piece | Top 26 cm half cut inner | 61.46 |
| | (cm) | |
| | Top 26 cm half cut inner | 61.46 |
| | (cm) | |
| | Top connection piece (cm) | 1.79 |
| Bottom clamp piece | 26 cm Bottom Half Cut | 106.50 |
| | Right | |
| | 26 cm Bottom Half Cut | 106.48 |
| | Right (cm) | |
| Pin | Double Pin Right 33 (cm) | 64.24 |
| | Double Pin Left 33 cm (cm) | 61.99 |

| | |
|---|---|
| Total | 463.92 |

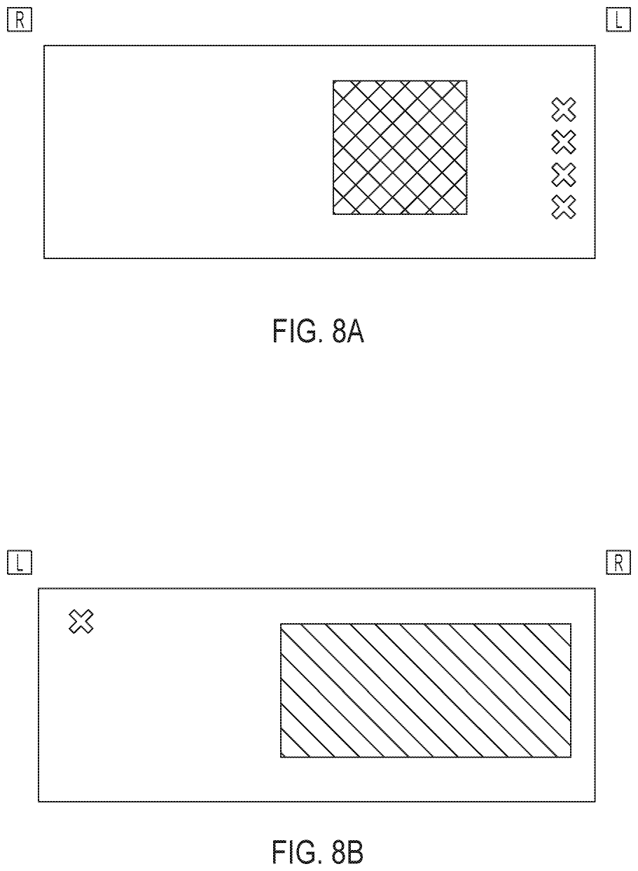

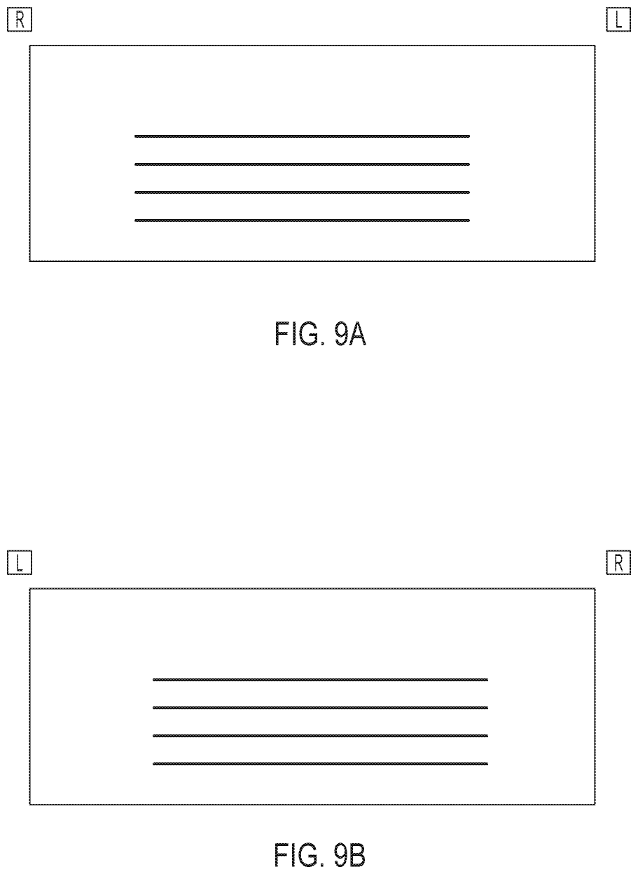

8A is a side view illustrating one embodiment of an outer cuff front panel on its non-TPU coated side of the present disclosure. FIG. 8B is a side view illustrating one embodiment of an outer cuff back panel on its non-TPU coated side of the present disclosure. FIG. 9A is a side view illustrating one embodiment of an outer cuff front panel on its TPU coated side of the present disclosure. FIG. 9B is a side view illustrating one embodiment of an outer cuff back panel on its TPU coated side of the present disclosure.

The geometry of the outer cuff, in the example embodiments provided by FIGS. 8A and 8B, is 65 cm in length by 30 cm in width, designed to comfortably accommodate a wide range of arm sizes. The outer cuff consists of two large panels of TPU-coated nylon. A TPU coating on the outer cuff provides enhanced durability, fluid resistance, weldability, and dimensional stability while permitting RF-welded seams and improved hygiene. The hook side of the hook and loop fastener is placed on the front panel, where the hook side, in one example embodiment, is 15 cm in length by 15 cm in width. The loop side of the hook and loop fastener is placed on the back panel. The orientation is detonated as L for left and R for right when looking at the blood pressure cuff from the back panel. Additionally, a hole is to be cut, denoted as an X, for the exterior tubing to be connected to the interior tubing system.

The outer cuff also includes internal dividers to both maintain the positioning of each air bladder and restrict the width of the air bladders while they inflate to the correct size. The dividers measure 40 cm by 6 cm. Additionally, the dividers limit the blood pressure cuff's inflated height to 4 cm.

In some embodiments, the outer cuff is constructed from 70D TPU-coated woven nylon, selected for its durability and biocompatibility. The outer cuff components are to be sewn together with a 2 cm seam 122, 124. Zigzag stitches are used for all outer seams, to increase the strength of the seam. Straight stitches are used to secure the dividers, as the accuracy of both the final divider height and the spacing between them is vital. The use of a straight stitch allows for greater precision. Additionally, small holes in the outer cuff allow the handles of the stopcocks 142, 148, 154, 160 to be accessible by the operator. These holes are spaced 2 cm apart, 3 cm from the edge of the material.

There are five different images that will be ultraviolet (UV) printed onto the blood pressure cuff. The first image includes labels and arrows that direct the operator in the orientation of the blood pressure cuff during placement. Each label is 2 cm by 3 cm in size.

The second image is a sizing guide to aid the operator in proper adjustment of the air bladder length located on the inside of the blood pressure cuff. When the operator holds the blood pressure cuff up behind the patient's arm, just beneath the deltoid muscle, the location of the patient's upper arm midpoint falls within the size range. If the patient's approximate midpoint lies on the border of two sizes, the higher number is selected. Each colored bar is 2 cm tall and 15 cm wide, for a total area of 8 cm by 15 cm. The distance from the top edge of the blood pressure cuff to the top of bar 1 is 14 cm.

The third image provides the valve numbers that correspond to the sizing guide from the second image. These numbers are located on the outer cuff. For the selected size, the selected valve is turned to the open position, as well as any valves with smaller numbers. Any valves with larger numbers than the selected valve will be turned to the off position. Each of these four numbers measure 1 cm by 1 cm.

The fourth image gives the location for the operator to align the brachial artery with.

The fifth image indicates if the stopcocks 142, 148, 154, 160 are in the open or closed position, and are 2 cm by 2 cm. The stopcocks 142, 148, 154, 160 include a handle with a green T for open and red T for closed. Each of these images are located underneath the handles of each of the stopcocks 142, 148, 154, 160.

### Outer Cuff Manufacture

First, TPU-coated nylon sheets will be cut to size using a die cutter. Two sizes will be cut, which include the 65 by 30 cm panels and the 40 by 6 cm dividers. Then, the panels will have the images UV printed on, as UV printing is precise, efficient, and cost effective for mass production. Additionally, the holes 104, 108 for the stopcock 142, 148, 154, 160 handles will also be cut.

Next, the hook and loop fasteners must be cut to size, with the hook section measuring 15 by 15 cm and the loop section measuring 40 by 15 cm. Both of these hook and loop fastener sections will be sewn onto their corresponding panels, following the dimensions using a zigzag stitch, as it allows the material to stretch without breaking the tread.

Next, one of the 65 cm length sides will be sewn to connect the two panels together. Each panel gives a seam allowance of 2 cm. Similar to the hook and loop fastener, this will also be sewn using a zigzag stitch.

The next step is to attach the dividers within the blood pressure cuff. First, each of the four dividers is sewn to the polyurethane side of the front panel of the blood pressure cuff using a straight stitch. Then, the opposite side of the dividers is sewn to the back panel. A 1 cm seam allowance is given on both sides of the divider, so the height of the divider once sewn is 4 cm.

Then the 30 cm side of the panels, opposite of the location of the tubing system, can be sewn together using a zigzag stitch, again with a 2 cm seam allowance.

Unlike the previous two side seams, which could be sewn inside out and righted by inverting the blood pressure cuff, the second 65 cm side can no longer be inverted because of the dividers within the blood pressure cuff. Therefore, the sewing methodology for this seam differs. Each of the edges will be tucked within the blood pressure cuff and then a zigzag stitch will be used to sew these together on the outside of the blood pressure cuff. Additional material length is given in this dimension to allow for this sewing technique, allowing a seam allowance of up to 10 cm.

Before the remaining 30 cm side can be sewn, the components of the blood pressure cuff must be inserted. This includes the five total air bladders and tubing system. Each of the handles of the stopcocks will be pushed through the holes in the outer cuff The air bladders will be inserted into each of their corresponding openings between the dividers. Additionally, the tubing and pressure valve will be connected to the additional connection port on the large air bladder. The tubing will be secured in place by sewing small pieces of nylon around the various tubing sections. This material is taken from the scrap material that occurs when die cutting the air bladder sections. Once all components are attached, and the connections verified to be airtight, the remaining 30 cm side can be sewed closed following the sewing methodology from the second 65 cm side, using a zig zag stitch.

Last, the clamp is secured to the blood pressure cuff by feeding the blood pressure cuff between the top and bottom clamp pieces and placing the pins in the holes. All manufacturing steps include quality control measures to detect and correct defects.

### Example 1

Table 3 gives the comparison between the required and the actual minimum and maximum patient arm circumferences. From this, it is determined that the blood pressure cuff design fits a wider range of patient arm circumferences than required.

TABLE 3
Patient Mid-Upper Arm Circumference Results

| | Minimum Patient Arm | Maximum Patient Arm |
|---|---|---|
| | Circumference (cm) | Circumference (cm) |
| Required | 25.6 | 42.8 |
| Actual | 25 | 54 |

### Example 2

Table 4 gives the comparison between the required and the actual minimum and maximum air bladder circumferences. From this, it is determined that the blood pressure cuff meets the air bladder sizing requirements exactly.

TABLE 4
Air Bladder Circumference Results

| | Minimum Air Bladder | Maximum Air Bladder |
|---|---|---|
| | Circumference (cm) | Circumference (cm) |
| Required | 20 | 35 |
| Actual | 20 | 35 |

### Example 3

Table 5 gives the maximum deflation rate tested using a tube with a diameter of 8.5 cm to mimic an arm at the smaller end of the arm circumference range, and using the largest circumference and length sizes. When the starting pressure was reached, the pressure release valve on the bulb pump was fully opened, and allowed to stay fully opened throughout the entirety of the test, which resulted in the maximum deflation rate that can occur.

TABLE 5
Air Average Air Bladder Deflation
Rate Results, Pressure Release Valve Open

| | Initial | Starting | | Deflation | |
|---|---|---|---|---|---|
| | Pressure | Pressure | Time | Rate | |
| Test | (mmHg) | (mmHg) | (s) | (mmHg/s) | |
| 1 | 0 | 100 | 7.96 | 12.56 | |
| 2 | 0 | 100 | 6.88 | 14.53 | |
| 3 | 0 | 100 | 7.76 | 12.89 | |
| 4 | 0 | 100 | 8.71 | 11.48 | |
| 5 | 0 | 100 | 8.76 | 11.42 | |
| 6 | 0 | 100 | 8.46 | 11.82 | |
| 7 | 0 | 100 | 9.26 | 10.80 | |
| 8 | 0 | 100 | 8.71 | 11.48 | |
| 9 | 0 | 100 | 8.35 | 11.98 | |
| 10 | 0 | 100 | 9.11 | 10.98 | |
| Average: | | | | 11.99 | mmHg/s |
| Standard Deviation: | | | | 1.10 | mmHg/s |
| 1 | 0 | 150 | 9.66 | 15.53 | |
| 2 | 0 | 150 | 9.28 | 16.16 | |
| 3 | 0 | 150 | 9.06 | 16.56 | |
| 4 | 0 | 150 | 8.80 | 17.05 | |
| 5 | 0 | 150 | 9.65 | 15.54 | |
| 6 | 0 | 150 | 9.03 | 16.61 | |
| 7 | 0 | 150 | 9.65 | 15.54 | |
| 8 | 0 | 150 | 9.38 | 15.99 | |
| 9 | 0 | 150 | 9.68 | 15.50 | |
| 10 | 0 | 150 | 9.15 | 16.39 | |
| Average: | | | | 16.09 | mmHg/s |
| Standard Deviation: | | | | 0.56 | mmHg/s |
| 1 | 0 | 200 | 10.86 | 18.42 | |
| 2 | 0 | 200 | 11.45 | 17.47 | |
| 3 | 0 | 200 | 11.35 | 17.62 | |
| 4 | 0 | 200 | 9.30 | 21.51 | |
| 5 | 0 | 200 | 10.93 | 18.30 | |
| 6 | 0 | 200 | 11.73 | 17.05 | |
| 7 | 0 | 200 | 9.31 | 21.48 | |
| 8 | 0 | 200 | 9.65 | 20.73 | |
| 9 | 0 | 200 | 8.76 | 22.83 | |
| 10 | 0 | 200 | 10.71 | 18.67 | |

| | | |
|---|---|---|
| Average: | 19.41 | mmHg/s |
| Standard Deviation: | 2.04 | mmHg/s |

### Example 4

Table 6 gives the results of the average air bladder inflation time testing. Three separate tests, with final test pressures of 100, 150, and 200 mmHg, were conducted. The test was conducted on a tube with a diameter of 8.5 cm to mimic an arm at the smaller end of the arm circumference range. Both the 100 mmHg and 150 mmHg testing results fall below the target value, with averages of 9.98 sand 11.21 s, respectively. While the 200 mmHg test did take an average of 15.16 s, this is not significantly larger than the target value. Furthermore, a hypertensive crisis is classified with a systolic pressure greater than 180 mmHg, where a medical professional is recommended to be contacted immediately Therefore, it is more likely that in daily use this higher pressure is not used as frequency and this marginal additional inflation time is justified.

TABLE 6
Average Air Bladder Inflation Time Results

| | Initial Pressure | Final pressure | Time | |
|---|---|---|---|---|
| Test | (mmHg) | (mmHg) | (s) | |
| 1 | 0 | 100 | 13.34 | |
| 2 | 0 | 100 | 9.78 | |
| 3 | 0 | 100 | 9.90 | |
| 4 | 0 | 100 | 9.76 | |
| 5 | 0 | 100 | 9.68 | |
| 6 | 0 | 100 | 10.93 | |
| 7 | 0 | 100 | 8.65 | |
| 8 | 0 | 100 | 9.66 | |
| 9 | 0 | 100 | 9.25 | |
| 10 | 0 | 100 | 8.86 | |
| Average: | | | 9.98 | s |
| Standard Deviation: | | | 1.33 | s |
| 1 | 0 | 150 | 12.10 | |
| 2 | 0 | 150 | 12.01 | |
| 3 | 0 | 150 | 10.40 | |
| 4 | 0 | 150 | 10.39 | |
| 5 | 0 | 150 | 10.20 | |
| 6 | 0 | 150 | 10.66 | |
| 7 | 0 | 150 | 11.75 | |
| 8 | 0 | 150 | 11.55 | |
| 9 | 0 | 150 | 11.65 | |
| 10 | 0 | 150 | 11.37 | |
| Average: | | | 11.21 | s |
| Standard Deviation: | | | 0.72 | s |
| 1 | 0 | 200 | 14.17 | |
| 2 | 0 | 200 | 16.42 | |
| 3 | 0 | 200 | 14.33 | |
| 4 | 0 | 200 | 15.75 | |
| 5 | 0 | 200 | 15.55 | |
| 6 | 0 | 200 | 13.71 | |
| 7 | 0 | 200 | 14.80 | |
| 8 | 0 | 200 | 14.96 | |
| 9 | 0 | 200 | 15.20 | |
| 10 | 0 | 200 | 16.66 | |

| | | |
|---|---|---|
| Average: | 15.16 | s |
| Standard Deviation: | 0.96 | s |

As the device must be able to fit a wide range of individuals, there are two main dimensions that are vital in blood pressure cuff sizing: the air bladder length and the arm circumference. For an upper arm blood pressure cuff, this translates to analysis of the upper arm length and mid-upper arm circumference values of the adult population. The blood pressure cuff illustrated herein can encompass 80% of the target population.

To quantify the targeted range of arm sizes, anthropometric dimension percentiles of U.S. adults were analyzed. It is common to set the design range between the smallest female (typically 5th percentile) and the largest male (typically 95th percentile), to cover 90% of the population. Therefore, to include both 80% of the population and include more the upper range, the 10th percentile for females and the 95th percentile for males was used, increasing to 85% of the population. The anthropometric U.S. adult percentile data from 2007-2010 and 2015-2018 were both found, and the larger upper percentile value and smaller lower percentile value were used to set the critical design requirement. This resulted in a target upper arm length of 33 cm to 42.9 cm and mid-upper arm circumference of 25.6 cm to 42.8 cm. The goal coverage length is defined as having an air bladder circumference that is at least 80% of the patient's arm circumference. Therefore, the air bladder design should have a circumference range of 20.48 to 34.24 cm, rounded to 20 to 35 cm.

The goal coverage length is defined as having an air bladder length that is 40% of the patient's upper arm length, and approximately 40% of the patient's arm circumference. This gives two ranges of air bladder length ranges: 13.2 to 17.16 cm and 10.24 to 17.12 cm. Combining these ranges, gives a range of 10.24 to 17.16 cm, rounded to 10 to 18 cm.

It should be understood that various changes and modifications to the presently preferred embodiments described herein will be apparent to those skilled in the art. It is therefore intended that any or all of such changes and modifications may be covered by the appended claims.

The invention is claimed as follows:

1. A blood pressure measuring device, comprising:
a cuff including a plurality of segments disposed along a first direction;
a plurality of air bladders disposed in the plurality of segments,
wherein the plurality of air bladders are configured to be selectively inflated such that a length of an inflated portion of the plurality of air bladders in the first direction is adjustable; and
a flow control device configured to be selectively coupled to a portion of the cuff, wherein the flow control device is configured to prevent air flow such that a circumference of the inflated portion of the plurality of air bladders is adjustable when the cuff is wrapped around an arm of a patient.

2. The blood pressure measuring device of claim 1, further comprising: a tubing system configured to selectively provide air to the plurality of air bladders.

3. The blood pressure measuring device of claim 2, wherein the tubing system includes a plurality of valves configured to selectively allow or prevent airflow to the plurality of air bladders, respectively.

4. The blood pressure measuring device of claim 3, wherein the plurality of valves includes a stopcock.

5. The blood pressure measuring device of claim 1, wherein the plurality of air bladders includes a first air bladder and a second air bladder, and wherein the first air bladder has a greater length in the first direction than the second air bladder.

6. The blood pressure measuring device of claim 1, wherein each segment of the plurality of segments of the cuff is configured to limit a height and a length in the first direction of a respective air bladder of the plurality of air bladders disposed therein when inflated.

7. The blood pressure measuring device of claim 3, wherein the air bladders are fluidly coupled in series along the first direction.

8. The blood pressure measuring device of claim 2, wherein the plurality of segments are arranged linearly along the first direction corresponding to a longitudinal axis of the arm.

9. The blood pressure measuring device of claim 1, wherein the flow control device is configured to compress the cuff to block airflow within the plurality of air bladders beyond the portion of the cuff to which the flow control device is coupled.

10. The blood pressure measuring device of claim 1, wherein the flow control device is movable along the first direction to select the circumference of the inflated portion.

11. The blood pressure measuring device of claim 1, wherein the flow control device is a clamp and wherein the clamp includes opposing clamp members configured to engage opposite sides of the cuff.

12. The blood pressure measuring device of claim 1, wherein the cuff includes an outer fabric layer configured to house the plurality of air bladders.

13. The blood pressure measuring device of claim 1, wherein the plurality of air bladders include a polymer material comprising a thermoplastic polyurethane-coated fabric.

14. The blood pressure measuring device of claim 1, wherein the flow control device defines a two-dimensional adjustment of the inflated portion of the cuff.

15. The blood pressure measuring device of claim 1, wherein each of the plurality of segments has a length in the first direction of about 1 cm to about 3 cm.

16. The blood pressure measuring device of claim 1, wherein each of the plurality of segments has a length of about 2 cm.

17. The blood pressure measuring device of claim 1, wherein the inflated portion of the plurality of air bladders has an adjustable circumference of about 20 cm to about 35 cm when wrapped around the arm of the patient.

18. The blood pressure measuring device of claim 1, wherein the inflated portion of the plurality of air bladders is configured to cover at least about 80% of a circumference of the arm of the patient.

19. The blood pressure measuring device of claim 11, wherein the clamp has a length in the first direction of about 25 cm to about 40 cm.

20. The blood pressure measuring device of claim 11, wherein the clamp has a length of about 36 cm.
